(19) United States
(12) Patent Application Publication
(10) Pub. No.: US 2009/0028898 A1
(43) Pub. Date: Jan. 29, 2009

(54) Instant Additive Solution for the Filter of Cigarette, Its Production Method and Use

(76) Inventors: Xixian Qiu, Jiangsu (CN); Xin Yu, Hubei (CN)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 11/886,379

(22) PCT Filed: Mar. 14, 2005
(86) PCT No.: PCT/CN2005/000304

§ 371 Date: Sep. 14, 2007

(51) Int. Cl. A61K 36/02; A61K 36/00; A61P 39/00

(52) U.S. Cl. 424/195.17; 131/200; 424/725

(57) ABSTRACT

The present invention relates to the field of tobacco science and smoking, providing an instant additive solution for the filter of cigarette which can efficiently reduce the tar and a great number of toxicants in main-stream smoke and simultaneously lower the temperature of main-stream smoke when flowing through the respiratory tract. Said instant additive solution for the filter of cigarette comprises following components: 1-500 nzg/mL of Vitamins, 1-300 mg/mL of tea polyphenols, and 5-200 mg/mL of the extracts of plants/algae. The present invention also relates to methods for preparing and using the instant additive solution for the filter of cigarette, as well as cigarette products comprising the instant additive solution for the filter of cigarette.

### FIELD OF THE INVENTION

The present invention relates to the field of tobacco science and smoking. Specifically, the present invention relates to an instant additive solution for the filter of cigarettes and methods for preparing and using the instant additive solution, as well as cigarette products comprising the instant additive solution for the filter of cigarettes.

### BACKGROUND OF THE INVENTION

Cigarettes produce a great number of gas toxicants in the combustion process due to thermal cracking, carbonization, incomplete combustion and evaporation. Among these toxicants, tar is formed due to incomplete combustion of organic components in cigarettes, e.g. cellulose, and carbonization. Up to date, many components in main-stream smoke are identified to be harmful. These components include substances carcinogenic or potentially carcinogenic to human, such as benzene, 2-naphthylamine, 4-amino-biophenyl, radioactive element Po-210, aldehyde, hydrazine, volatile nitrosamine, nitrosamine specific in cigarettes, N-nitroso-pyridine, benzo[α]pyrene, N-nitrosodiethylamine and cadmium, as well as known potential carcinogens to animals, such as benzo [α]anthracene, butyrolactone, N-nitrosonomicotine. All of the above substances have direct toxicity to somatic cells.

At present, tar and toxicants reducing techniques commonly used in tobacco industry include:
1. Modify tobacco leaf formula composition, produce blending type cigarettes;
2. Improve tobacco shred and tobacco sheet during the manufacturing process of cigarettes, so as to improve the combustion of cigarettes. For example, add combustion-supporting agents, e.g. K, into tobacco shred; improve the additives to cigarette bobbin paper, e.g., cigarette bobbin paper prepared by mixing potassium citrate, calcium carbonate and wood fiber has better permeability and higher static combustion rate; expand tobacco shred and expand cut stem. Therefore, the tar content derived from incomplete combustion may be reduced significantly. In addition, tar content may also be reduced by improving the adulterated tobacco sheets. For example, add zeolites that have selective absorbable and catalytic effects on tobacco to tobacco sheets; add 10-18% sepiolite powder before drying tobacco sheets. These methods by which substances are added in tobacco shreds or tobacco sheets are effective in reducing toxicants in cigarettes.
3. Develop flavoring and casing technology, develop new flavors, so as to match the needs to reduce tar content in cigarettes.
4. Research and develop special cigarettes without tobacco shred combustion;
5. Use the filter of cigarettes. By connecting various filters to cigarettes, including ordinary filter, filter produced with special filter material and complex filter, tar content may be reduced by 20-50%. This method is most widely used in the world to reduce tar content.

The filter of cigarettes produced with acetate fiber, man-made fiber, cotton and paper reduces the particles in smoke by mechanical effects. It may also reduce gas substances in smoke. Currently, approximately 90% filters of cigarettes are composed of single acetate fiber. Although this kind of filter may remove a great amount of toxicants from smoke, it inevitably allows substantial toxicants to go into human body. Improving this kind of filter, such as increasing the number of crimps on the surface of fiber, extending specific surface area and applying some additives, are effective in further reducing toxicants in smoke. Among various additives used in the filters of cigarettes, a major category of additives is porous sorbents, such as active carbon, sepiolite, montmorillonite, maifan stone, silicone, hydroxyapatite, organic silicon, synthetic mica, hemachrome, chitosan, anion exchange resin and cation exchange resin, etc. These sorbents are often used as additives in the complex filters of cigarettes. In addition, tea, coffee, lichen and porphyrin complex are also used as additives. The filters of cigarettes have greatly developed. The types of filters have developed from ordinary acetate fiber to environment-protective biodegradable, and complex containing additives filters, from ordinary shape and functions to special shape filters, such as hollow complex filters of cigarettes, heart-shaped filters of cigarettes. These filters of cigarettes mainly remove or reduce the following toxicants: HCN, poly aromatic hydrocarbon, free radicals, nucleophilic toxins, tar, nicotine and smoke condensates, etc. For example, perforated filters of cigarettes may not only dilute cigarette smoke, but also reduce CO content in lateral main-stream smoke efficiently.

In the research of reducing tar and toxicants in cigarettes, the inventors found that although the above methods may reduce the tar and toxicants in main-stream of smoke, their effects are usually not satisfactory. The additives in complex filters of cigarettes have been shown not to be always effective through the smoking process. Obviously, it is related to physical and chemical properties of the additives. For example, although some additives have a large specific surface area, the actual utilization ratio is relatively low. As a result, some highly active substances, such as free radicals, may quickly go through the filter of cigarette into the respiratory tract at a high flow rate of main-stream smoke. Similarly, since the specific surface area of acetate fiber is only about 4.5 m²/g, it is difficult to adsorb a great amount of toxicant on the surface. Furthermore, after the additives are added into the filters of cigarettes, they will go through a period of storage or circulation until they are used. During this period, the additives would be contaminated by some volatilized substances from the cigarette shreds. Subsequently, the effectiveness of the additives would be reduced significantly.

In summary, no effective method to reduce the tar and a great number of toxicants in main-stream smoke and simultaneously lower the temperature of main-stream smoke when flowing through the respiratory tract has been found up to date due to the above limitations existing in current technologies. Furthermore, there is little variation in cigarette taste. Therefore, there is an urgent need in this field for developing a cost-effective method to reduce the tar and a great number of toxicants in main-stream smoke and simultaneously lower the temperature of main-stream smoke when flowing through the respiratory tract.

### SUMMARY OF THE INVENTION

The objective of the present invention is to provide an instant additive solution for the filter of cigarette that has solved the above technical problems, and methods for preparing and using the instant additive solution.

In one aspect, the present invent provides an instant additive solution for the filter of cigarette containing the following components: 1-500 mg/ml of Vitamins, 1-300 mg/ml of tea polyphenols, and 5-200 mg/ml of the extracts of plants/algae.

In a preferred embodiment, the said instant additive solution for the filter of cigarette contains 2-400 mg/ml of Vitamins, 2-250 mg/ml of tea polyphenols, and 10-175 mg/ml of the extracts of plants/algae.

In another preferred embodiment, the said instant additive solution for the filter of cigarette contains sodium selenite, bee propolis and cysteine in addition to the above basic components.

In another preferred embodiment, the said Vitamins are selected from the group consisting of Vitamin C, Vitamin E and their combinations; the said extracts of plants/algae are selected from the group consisting of the extracts of grape seeds, the extracts of pine bark, the extracts of tea, the extracts of algae and their combinations.

In another preferred embodiment, the said instant additive solution for the filter of cigarette contains trace elements in addition to the above basic components. The said trace elements are selected from the group consisting of selenium, zinc, calcium, iron and their combinations.

In another preferred embodiment, the said instant additive solution for the filter of cigarette contains flavor component in addition to the above basic components.

In a further aspect, the present invention provides a method for preparing the instant additive solution for the filter of cigarette, which includes: add vitamins, tea polyphenols and the extracts of plants/algae to water, thus obtain solutions of 1-500 mg/ml of Vitamins, 1-300 mg/ml of tea polyphenols, and 5-200 mg/ml of the extracts of plants/algae.

In a further aspect, the present invention also provides a method for using the instant additive solution for the filter of cigarette, which includes: add 10-500 μl of the said instant additive solution for the filter of cigarette to the filter of cigarette 0-30 minutes before smoking cigarette.

In a further aspect, the present invention also provides another method for using the instant additive solution for the filter of cigarette, which includes: prepare a separate product of the said instant additive solution for the filter of cigarette, and then put the product in the filter for cigarette or cigarette package as a separate additive for the filter of cigarette.

In still a further aspect, the present invention also provides a cigarette product, which includes: a cigarette package container, 5-100 cigarettes in the said container, a sealed container for the said instant additive solution for the filter of cigarette that can be opened, as well as 0.5-50 ml of the said instant additive solution for the filter of cigarette in the said sealed container.

### DETAILED DESCRIPTION OF THE EMBODIMENTS

In the present invention, the instant additive solution for the filter of cigarette is added separately 0-30 minutes before smoking cigarette, so it is independent from cigarette. After appropriate amount of the said solution is added to the filter tow of cigarette, the surface of the filter tow will become wet, thus providing a cooling effect by which the solution can efficiently lower the temperature of main-stream smoke when flowing through the respiratory tract, reduce the injury to the mucous membrane covering the respiratory tract caused by high temperature, as well as condense quickly the trace toxicants with a high boiling point in main-stream smoke, such as polynuclear aromatic hydrocarbon; the lower temperature can also reduce the diffusion of toxicants in main-stream smoke, thus further reducing the injury to the mucous membrane; since the instant additive solution for the filter of cigarette contains anti-oxidative components, it can efficiently capture the free radicals and other highly oxidative components in main-stream of smoke, as well as remove nucleophilic toxins in main-stream smoke; when the solution contains Vitamin E, adding selenium to the solution can strengthen the effect; a film formed by water solution can efficiently dissolve water-soluble toxins to cilia in mainstream smoke, such as HCN and acrolein. The instant additive solution for the filter of cigarette can be designed as a flavor adding or transmitting solution, so as to improve the smoking taste.

In the present invention, the instant additive solution for the filter of cigarette contains solvents and active ingredients which can reduce toxicants.

The solvents that can be used in the present invention include water or aquatic solvents. The said water can be purified water, distilled water, underground water or tap water. In a broad sense, the said water can also include aquatic solvents, i.e. mixture of more than 80% of water and other solvents, e.g. alcohol.

The active components in the present invention mainly include Vitamins, tea polyphenols and extracts of plants/algae.

Vitamins that can be used in the present invention include vitamin C and vitamin E. The content is usually 1-500 mg/ml; 2-400 mg/ml is preferable; 200 mg/ml is even better.

The content of tea polyphenols used in the present invention is usually 1-300 mg/ml; 2-250 mg/ml is preferable; 125 mg/ml is even better.

The extracts of plants/algae that can be used in the present invention include the extracts of grape seeds, the extracts of pine bark, the extracts of tea and the extracts of algae. The content is usually 5-200 mg/ml; 10-175 mg/ml is preferable; 125 mg/ml is even better.

In the present invention, the instant additive solution for the filter of cigarette can also contain sodium selenite, bee propolis and cysteine. The content is usually 0-100 mg/ml; 0.1-50 mg/ml is preferable.

In the present invention, the instant additive solution for the filter of cigarette can also contain trace elements. The said trace elements are selected from selenium, zinc, calcium, iron. The content is usually 0.001-1 mg/ml; 0.05 mg/ml is preferable. The instant additive solution for the filter of cigarette can also contain other additives, such as anti-oxidants, preservatives, pigment, wetting agents, flavors (e.g. mint and other flavors commonly used in cigarettes). The content is usually 0.1-50 mg/ml; 1-10 mg/ml is preferable.

The present invention provides a method for preparing the instant additive solution for the filter of cigarette, which includes: add vitamins, tea polyphenols and the extracts of plants/algae to water or aquatic solvents, thus obtain an instant additive solution for the filter of cigarette containing 1-500 mg/ml of Vitamins, 1-300 mg/ml of tea polyphenols, and 5-200 mg/ml of the extracts of plants/algae. The active components can be added simultaneously or sequentially. Adding the components in stirring process is preferred.

The present invention also provides a method for using the instant additive solution for the filter of cigarette, which includes: add 10-500 μl of the said instant additive solution for the filter of cigarette to the filter of cigarette 0-30 minutes before smoking cigarette.

In a preferred embodiment, prepare a separate product of the said instant additive solution for the filter of cigarette, then put the product in the filter of cigarette or cigarette package as a separate additive for the filter of cigarette.

The present invention also provides a cigarette product, which includes: a cigarette package container, 5-100 cigarettes in the said container, a sealed container for the said instant additive solution for the filter of cigarette that can be opened, as well as 0.5-50 ml of the said instant additive solution for the filter of cigarette in the said sealed container.

In one preferred embodiment, the said sealed container is located outside the cigarette package container. In another preferred embodiment, the said sealed container is located inside the cigarette package container. Even better, the shape of said sealed container is the same as that of cigarette.

The advantages of the present invention include efficiently reducing the tar and a great number of toxicants in main-stream smoke, such as acrolein, cyanide, sinapylaldehyde, nicotine, nitrosamine, some polynuclear aromatic hydrocarbons (e.g. benzo[α]pyrene) and free radicals, and simultaneously lowering the temperature of main-stream smoke when flowing through the respiratory tract, minimizing the injuries to smokers caused by smoking.

The following embodiments are for illustrative purpose and are not intended to limit the scope of the invention.

### Embodiment 1

Weigh 10 mg Vitamin C and dissolve in 5 ml purified water. Weigh 10 mg tea polyphenol and 50 mg tea extracts, respectively, add to the above solution, stir until dissolved. Dilute to volume 10 ml, thus obtain an instant additive solution for the filter of cigarette. The concentrations of Vitamin C, tea polyphenols and tea extracts in the solution are 1 mg/ml, 1 mg/ml and 5 mg/ml, respectively. While smoking cigarette, add 10 μl of the said instant additive solution to the filter, so as to improve the smoking taste. The assessment after smoking is that the solution reduces significantly the stimulation of mainstream smoke and produces a more gentle and favorable taste.

### Embodiment 2

Weigh 5,000 mg Vitamin E and dissolve in 5 ml purified water. Weigh 3000 mg tea polyphenol and 2,000 mg extracts of grape seeds, respectively, add to the above solution, stir until dissolved. Dilute to volume 10 ml, thus obtain an instant additive solution for the filter of cigarette. The concentrations of Vitamin E, tea polyphenol and grape seeds extracts in the solution are 500 mg/ml, 300 mg/ml and 200 mg/ml, respectively. Add 500 μl of the said instant additive solution to the filter 30 minutes before smoking cigarette, so as to improve the smoking taste. The assessment after smoking is that the solution reduces significantly the stimulation of mainstream smoke and produces a more gentle and favorable taste.

### Embodiment 3

Weigh 20 mg Vitamin C and dissolve in 5 ml purified water. Weigh 20 mg Vitamin E, 20 mg tea polyphenol and 100 mg pine bark extracts, respectively, add to the above solution, stir until dissolved. Dilute to volume 10 ml, thus obtain an instant additive solution for the filter of cigarette. The concentrations of Vitamin C, Vitamin E, tea polyphenol and pine bark extracts in the solution are 2 mg/ml, 2 mg/ml, 2 mg/ml and 10 mg/ml, respectively. Add 250 μl of the said instant additive solution to the filter 15 minutes before smoking cigarette, so as to improve the smoking taste. The assessment after smoking is that the solution reduces significantly the stimulation of mainstream smoke and produces a more gentle and favorable taste.

### Embodiment 4

Weigh 4,000 mg Vitamin C and dissolve in 5 ml purified water. Weigh 4,000 mg Vitamin E, 2,500 mg tea polyphenol, 1,750 mg algae extracts and 500 mg sodium selenite, respectively, add to the above solution, stir until dissolved. Dilute to volume ml, thus obtain an instant additive solution for the filter of cigarette. The concentrations of Vitamin C, Vitamin E, tea polyphenol, algae extracts and sodium selenite in the solution are 400 mg/ml, 400 mg/ml, 250 mg/ml, 175 mg/ml and 50 mg/ml, respectively. Add 200 μl of the said instant additive solution to the filter 5 minutes before smoking cigarette, so as to improve the smoking taste. The assessment after smoking is that the solution reduces significantly the stimulation of mainstream smoke and produces a more gentle and favorable taste.

### Embodiment 5

Weigh 1,000 mg Vitamin C and dissolve in 5 ml purified water. Weigh 1,000 mg Vitamin E, 1,000 mg tea polyphenol, 1,000 mg algae extracts and 250 mg bee propolis, respectively, add to the above solution, stir until dissolved. Dilute to volume 10 ml, thus obtain an instant additive solution for the filter of cigarette. The concentrations of Vitamin C, Vitamin E, tea polyphenol, algae extracts and bee propolis in the solution are 100 mg/ml, 100 mg/ml, 100 mg/ml, 100 mg/ml and 25 mg/ml, respectively. Add 350 μl of the said instant additive solution to the filter 10 minutes before smoking cigarette, so as to improve the smoking taste. The assessment after smoking is that the solution reduces significantly the stimulation of mainstream smoke and produces a more gentle and favorable taste.

### Embodiment 6

Weigh 3,000 mg Vitamin C and dissolve in 5 ml purified water. Weigh 3,000 mg Vitamin E, 1,500 mg tea polyphenol, 1,500 mg algae extracts and 50 mg cysteine, respectively, add to the above solution, stir until dissolved. Dilute to volume 10 ml, thus obtain an instant additive solution for the filter of cigarette. The concentrations of Vitamin C, Vitamin E, tea polyphenol, algae extracts and cysteine in the solution are 300 mg/ml, 300 mg/ml, 150 mg/ml, 150 mg/ml and 5 mg/ml, respectively. Add 150 μl of the said instant additive solution to the filter 20 minutes before smoking cigarette, so as to improve the smoking taste. The assessment after smoking is that the solution reduces significantly the stimulation of mainstream smoke and produces a more gentle and favorable taste.

### Embodiment 7

Weigh 2,000 mg Vitamin C and dissolve in 5 ml purified water. Weigh 2,000 mg Vitamin E, 1,250 mg tea polyphenol, 1,250 mg algae extracts, 200 mg sodium selenite, 300 mg cysteine and 0.1 mg calcium respectively, add to the above solution, stir until dissolved. Dilute to volume 10 ml, thus obtain an instant additive solution for the filter of cigarette. The concentrations of Vitamin C, Vitamin E, tea polyphenol, algae extracts, sodium selenite, cysteine and calcium in the solution are 200 mg/ml, 200 mg/ml, 125 mg/ml, 125 mg/ml, 20 mg/ml, 30 mg/ml and 0.01 mg/ml, respectively. Add 50 μl of the said instant additive solution to the filter 25 minutes before smoking cigarette, so as to improve the smoking taste. The assessment after smoking is that the solution reduces significantly the stimulation of mainstream smoke, produces a more gentle and favorable taste.

### Embodiment 8

Weigh 2,000 mg Vitamin C and dissolve in 5 ml purified water. Weigh 2,000 mg Vitamin E, 1,250 mg tea polyphenol, 1,250 mg algae extracts, 50 mg sodium selenite, 500 mg cysteine and 0.5 mg selenium respectively, add to the above solution, stir until dissolved. Add 1 ml saturated menthol solution, then dilute to volume 10 ml, thus obtain an instant additive solution for the filter of cigarette. The concentrations of Vitamin C, Vitamin E, tea polyphenol, algae extracts, sodium selenite, cysteine and selenium in the solution are 200 mg/ml, 200 mg/ml, 125 mg/ml, 125 mg/ml, 5 mg/ml, 50 mg/ml and 0.05 mg/ml, respectively. While smoking cigarette, add 400 μl of the said instant additive solution to the filter so as to improve the smoking taste. The assessment after smoking is that the solution reduces significantly the stimulation of mainstream smoke and produces a gentler and favorable taste, offers a cool and fresh feeling, resolves the problem of mint rapidly volatilizing while smoking mint cigarette without increasing technical difficulty.

### Embodiment 9

Weigh 2,000 mg Vitamin C and dissolve in 5 ml purified water. Weigh 2,000 mg Vitamin E, 1,250 mg tea polyphenol, 1,250 mg algae extracts, 1,250 mg tea extracts, 250 mg sodium selenite, 100 mg bee propolis, 300 mg cysteine, 0.1 mg zinc and 0.1 mg iron respectively, add to the above solution, stir until dissolved. Add 1 ml saturated menthol solution, then dilute to volume 10 ml, thus obtain an instant additive solution for the filter of cigarette. The concentrations of Vitamin C, Vitamin E, tea polyphenol, algae extracts, tea extracts, sodium selenite, bee propolis, cysteine, zinc and iron in the solution are 200 mg/ml, 200 mg/ml, 125 mg/ml, 125 mg/ml, 125 mg/ml, 25 mg/ml, 10 mg/ml, 30 mg/ml, 0.01 mg/ml and 0.01 mg/ml respectively. While smoking cigarette, add 500 μl of the said instant additive solution to the filter 30 minutes before smoking cigarette, so as to improve the smoking taste. The assessment after smoking is that the solution reduces significantly the stimulation of mainstream smoke and produces a gentler and favorable taste, offers a cool and fresh feeling, resolves the problem of mint rapidly volatilizing while smoking mint cigarette without increasing technical difficulty.

### Embodiment 10

Add no more than 10 ml of the instant additive solution for the filter of cigarette prepared as described above to the filter 15 minutes before smoking cigarette, then put in a smoking machine to perform standard smoking (inhale for 2 seconds, stop for 60 seconds). Then collect the tar on the Cambridge filter for analysis. The analysis results show that the said solution can reduce nicotine by more than 40%. Among the toxicants reduced by the said solution, acrolein and HCN are reduced by more than 95%, benzo[α]pyrene is reduced by more than 80%. For some cigarettes with complex filter, the said solution can make even greater improvement, achieving undetectable level of free radicals. The results are shown in table 1.

TABLE 1

| | | | | Decrease of |
|---|---|---|---|---|
| Embodi- | Decrease of | Decrease of | Decrease of | benzo[α]pyrene |
| ment | nicotine (%) | acrolein (%) | HCN(%) | (%) |
| 1 | >40 | >95 | >95 | >80 |
| 2 | >40 | >95 | >95 | >80 |
| 3 | >45 | >95 | >95 | >80 |
| 4 | >45 | >95 | >95 | >80 |
| 5 | >50 | >95 | >95 | >80 |
| 6 | >50 | >95 | >95 | >80 |
| 7 | >50 | >95 | >95 | >80 |
| 8 | >50 | >95 | >95 | >80 |
| 9 | >50 | >95 | >95 | >80 |

It can be understand that technical personnel may make various modification or revision to the present invention after reading the above text about the present invention. These equivalencies are within the scope defined in the claims of the application.

1. An instant additive solution for the filter of cigarette, containing 1-500 mg/ml of Vitamins, 1-300 mg/ml of tea polyphenols and 5-200 mg/ml of the extracts of plants/algae.

2. The instant additive solution for the filter of cigarette as in claim 1, wherein, it contains 2-400 mg/ml of Vitamins, 2-250 mg/ml of tea polyphenols and 10-175 mg/ml of the extracts of plants/algae.

3. The instant additive solution for the filter of cigarette as in claim 1, wherein, it also contains sodium selenite, bee propolis and cysteine.

4. The instant additive solution for the filter of cigarette as in claim 1, wherein, said Vitamins are selected from Vitamin C, Vitamin E and their combinations; the said extracts of plants/algae are selected from the extracts of grape seeds, the extracts of pine bark, the extracts of tea, the extracts of algae and their combinations.

5. The instant additive solution for the filter of cigarette as in claim 1, wherein, it also contains trace elements; said trace elements are selected from selenium, zinc, calcium, iron and their combinations.

6. The instant additive solution for the filter of cigarette as in claim 1, wherein, it also contains flavors.

7. A method for preparing the instant additive solution for the filter of cigarette as in claim 1, wherein, it includes: adding vitamins, tea polyphenols and the extracts of plants/algae to water respectively, thus to obtain solutions of 1-500 mg/ml of Vitamins, 1-300 mg/ml of tea polyphenols, and 5-200 mg/ml of the extracts of plants/algae.

8. A method for using the instant additive solution for the filter of cigarette as in claim 1, wherein, it includes: adding 10-500 μL of said instant additive solution for the filter of cigarette to the cigarette filter 10-30 minutes before smoking the cigarette.

9. A method for using the instant additive solution for the filter of cigarette as in claim 1, wherein, it includes: preparing a separate product of the said instant additive solution for the filter of cigarette, and then put the product in the filter for cigarette or cigarette package as a separate additive for the filter of cigarette.

10. A cigarette product, comprising: a cigarette package container, 5-100 cigarettes in the said container, a sealed container for the instant additive solution for the filter of cigarette as in claim 1 that can be opened, as well as 0.5-50 ml of the said instant additive solution for the filter of cigarette in the said sealed container.
